(19) United States
(12) Patent Application Publication
(10) Pub. No.: US 2004/0001834 A1
(43) Pub. Date: Jan. 1, 2004

(54) Phospholipase A2 enzyme, antibodies and inhibitors thereto

(76) Inventors: Dae-Kyong Kim, Seoul (KR); Chung-Kyu Ryu, Seoul (KR); Hae-Sook Shin, Gyeongsan-si (KR); Sung-Yun Jung, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/608,599

(22) Filed: Jun. 25, 2003

(30) Foreign Application Priority Data
Jun. 27, 2002 (KR) 10-2002-0036249

(51) Int. Cl. A61K 39/395; C12N 9/20; C07K 16/40

(52) U.S. Cl. 424/146.1; 435/198; 530/388.26

(57) ABSTRACT

The disclosed invention relates to a novel rPLA₂ enzyme, it's antibody and use and the preparation method. The purified enzyme, derived from RBC cytosol, produces arachidonic acid dependently to calcium ion, has 42 kDa of M.W. determined by SDS-PAGE analysis, shows its isoeletric point at the pH ranging 3.9 to about 4.1 in electrophoresis analysis, has maximum activity at the pH ranging 9.5 to 10 and has its specific activity of 5.6 nM/min/mg. The antibody of disclosed invention reacts with the enzyme rPLA₂ only and rPLA₂ is inactivated by EA4 compound, rPLA₂ inhibitor. The disclosed invention shows that the 42 kDa rPLA₂ identified as a novel form of Ca²⁺-dependent PLA₂ plays an important role in hemostasis, thrombosis and/or erythropoiesis through the Ca²⁺-dependent release of AA.

### RELATED APPLICATION

This application claims priority to Korean Patent Application No. KR10-2002-0036249, filed Jun. 27, 2002, which is hereby incorporated by reference in its entirety.

### FIELD OF THE INVENTION

The present invention relates to a novel phospholipase A₂ (PLA₂) enzyme, antibodies that react with the phospholipase A₂, enzyme inhibitor and use and the preparation method thereof.

### BACKGROUND OF THE INVENTION

Red blood cells (RBCs) enhance platelet aggregation in vitro induced by calcium ionophores, collagen, thrombin, shear stress and the like. Collagen-stimulated platelets aggregate three times more effectively and discharge seven times more adenosine diphosphate (ADP) in the present of RBCs than in the absence of RBCs. This increased activity shows that RBCs are clinically correlated with pathophysiological responses of platelets.

When RBCs are stimulated by the Ca²⁺ ionophore A23187 and shear stress, they release arachidonic acid (AA) from membrane phospholipids, possibly by the action of PLA₂ from cell membrane. There are several types of PLA₂, such as cPLA₂ (cytosolic phospholipase A₂) existing in cytosol and sPLA₂ (secretory phospholipase A₂) released to cell exterior. These enzymes have been isolated from various species. Paysant et al. detected PLA₂ activity in RBC membrane from rat and human (Paysant M. et al.; Bull. Soc. Chim. Biol. 52, pp1257-1269, 1970) and Kramer et al. described the purification of a calcium dependent 18.5-kDa PLA₂ from sheep RBC membrane (Kramer R. M. et al.; Biochim. Biophy. Acta, 507, pp381-394, 1978). Adachi et al. reported detecting a calcium-independent cytosolic PLA₂ preferentially hydrolyzing phosphatidylethanolamine to phosphatidylcholine in chicken RBCs (Adachi, I. Toyoshima, S. & Osawa, T.; Arch. Biochem. Biophys. 226, pp118-124, 1983). However, there has been no report concerning the antibody or inhibitor to specifically inhibit the activity this enzyme. Moreover, the RBC forms of PLA₂ have not been well studied.

The arachidonic acid released from RBCs is subsequently metabolized to eicosanoids. Eicosanoids dissociated by cyclooxygenase form prostaglandins. Prostaglandins interacting with platelets are then converted into thromboxanes. Eicosanoids dissociated by lipoxygenase form leucotrienes.

Eicosanoids play the role of messenger in various physiological functions and also take part in various phenomena such as hematopoiesis, the inflammation reaction, blood coagulation and control of blood pressure. Prostaglandins (PGs) are formed from almost all the cell in human body and stimulate blood constriction, alleviate pain resulting from an inflammation reaction and inhibit RBC volume regulation and filterability. Thromboxanes (TX) formed from platelets take part in forming thrombus comprising aggregating platelet and reduce the velocity of blood flow. Leucotrienes (LT) are formed from leucocytes and play a crucial role in the induction of inflammation and allergic responses.

From the discussion above it is clear that the eicosanoids play an indispensable regulatory role in vivo. However, overproduction of eicosanoids enhances platelet aggregation, results in the accumulation of thrombus, induces excessive constriction of blood vessel and finally induces various diseases such as arteriosclerosis, cerebral infarction, angina, cardiac infarction, chronic inflammation syndrome, other disorders of the immune system, cancer and the like.

### SUMMARY OF THE INVENTION

According to one aspect, the present invention provides a novel cytosolic PLA₂ enzyme isolated from RBC.

The present invention provides the antibody and inhibitor against the above enzyme.

The present invention also provides the preparation method for preparing above enzyme.

The present invention also provides a pharmaceutical composition comprising a therapeutically effective amount of antibody or inhibitor against rPLA₂ and a pharmaceutically acceptable carrier, for the treatment of diseases caused by disorder of hematopoietic system related to rPLA₂ enzyme.

Further, the present invention provides the method of treating various diseases related to Ca²⁺-dependent release of arachidonic acids comprising administering to said mammal an effective amount of an antibody or inhibitor set forth above with pharmaceutically acceptable carrier.

### BRIEF DESCRIPTION OF THE DRAWINGS

FIG. 1 shows release of [³H]AA by a calcium ionopore from human and bovine RBCs.

FIG. 2 represents PLA₂ activity of fractions from the butyl-toyopearl hydrophobic column chromatography in 1^{st} purification process of rPLA₂ from bovine RBCs and porcine spleen.

FIG. 3 represents PLA₂ activity of fractions from the phenyl-5PW hydrophobic HPLC column chromatography of aforementioned fraction.

FIG. 4 represents PLA₂ activity of fractions from the DEAE-5PW HPLC column chromatography of aforementioned fraction.

FIG. 5 represents PLA₂ activity of fractions from the superose 12 gel filtration FPLC column chromatography of aforementioned fraction.

FIG. 6 presents one-dimensional SDS-PAGE of final mono-Q FPLC elutes.

FIG. 7 presents two-dimensional SDS-PAGE of final mono-Q FPLC elutes.

FIG. 8 presents western blot analysis of rPLA₂ active pools with anti-cPLA₂ antibody.

FIG. 9 presents western blot analysis of rPLA₂ active pools with anti-sPLA₂ antibody.

FIG. 10 depicts the Lineweaver-Burk plot analysis of rPLA₂.

FIG. 11 depicts the substrate specificity of rPLA₂.

FIG. 12 depicts the calcium ion dependency on activity of rPLA₂.

FIG. 13 depicts the pH effect on the activity of rPLA₂.

FIG. 14 depicts the inhibition of the rPLA₂ activity with DTT.

FIG. 15 depicts the inhibition of the rPLA₂ activity with AACOCF₃.

FIG. 16 depicts the inhibition of the rPLA₂ activity with mepacrine.

FIG. 17 depicts the inhibition of the rPLA₂ activity with methyl mercury chloride.

FIG. 18 presents the immunoprecipitation analysis of rPLA₂.

FIG. 19 presents the remained PLA₂ activity of rPLA₂ immunoprecipitate with the lapse of time.

FIG. 20 presents the immunoprecipitation analysis of rPLA₂ with human phenyl-5PW fractions.

FIG. 21 presents the PLA₂ activity of human phenyl-5PW fractions.

FIG. 22 presents the effect of EPO on rPLA₂ expression.

FIG. 23 presents the DAB staining of MFL cells after 0 day culture.

FIG. 24 presents the DAB staining of MFL cells after 3 days culture in the absence of EPO.

FIG. 25 presents the DAB staining of MFL cells after 3 days culture in the presence of EPO.

FIG. 26 presents the DAB staining of MFL cells after 7 days culture in the absence of EPO.

FIG. 27 presents the DAB staining of MFL cells after 7 days culture in the presence of EPO.

FIG. 28 presents the immunoprecipitation analysis with rPLA₂ Ab of above-mentioned 5 kinds of MFL cells.

FIG. 29 presents the immunoprecipitation analysis with cPLA₂ Ab of above mentioned 5 kinds of MFL cells.

FIG. 30 presents the inhibition of rPLA₂ and cPLA₂ by quinone derivatives, TP1 and EA4.

FIG. 31 presents the determination of the inhibitory pattern on rPLA₂ by EA4.

FIG. 32 presents AA release induced by A23187, EA4, or TP1 in human RBCs.

FIG. 33 presents AA release induced by A23187, EA4 or TP1 in bovine RBCs.

FIG. 34 presents AA release induced by A23187, EA4 or TP1 in L929 cell.

### DETAILED DESCRIPTION OF THE PREFERRED EMBODIMENT

The present invention relates to a cytosolic 42-kDa calcium-dependent PLA₂. A bovine form of the enzyme was identified using biochemical and immunochemical studies and matrix-assisted laser desorption/ionization time-of flight (MALDI-TOF) mass spectrometric analysis. These studies have shown that the isolated and purified enzyme described herein is a novel phospholipase designated as rPLA₂.

Accordingly, one aspect of the present invention relates to a novel cytosolic PLA₂ enzyme isolated from RBCs. Another aspect of the present invention provides an antibody that reacts specifically with the novel enzyme and methods for generating the same. Additionally, another aspect of the present invention relates to the identification of an inhibitor of the novel enzyme.

The present invention provides methods for the preparation of the novel enzyme. Also, the present invention provides methods of treating various diseases related to the Ca²⁺-dependent release of arachidonic acids comprising administering to said mammal an effective amount of above antibody or inhibitor with pharmaceutically acceptable carrier.

One aspect of the present invention provides a novel cytosolic rPLA₂ enzyme derived from RBC cytosol characterized in that the enzyme produces arachidonic acid in a calcium dependent manner, has a molecular weight of approximately 42 kDa as determined by SDS-PAGE, has an isoeletric point from about 3.9 to 4.1, has a maximum activity at a pH from about 9.5 to about 10, and has its specific activity of about 5.6 nM/min/mg.

The cytosolic rPLA₂ enzyme described herein acts on the sn-2 position of a phospholipid and metabolizes the phospholipid to arachidonic acid (AA) in a calcium dependent manner. The cytosolic rPLA₂ enzyme prefers 2-AA-GPC (1-Stearoyl-2-arachidonyl-sn-glycerol-3-phosphocholine) among various phospholipids as a substrate. When the enzyme metabolizes 2-AA-GPC to form AA as a substrate, the Km and Vmax value of above enzyme are approximately 13.9 mM and 7.4 nM/min/mg, respectively. The described enzyme has a much lower specific activity (5.6 nmol/min/mg of protein) as compared to that of cPLA₂ (3,800-8,630 nmol/min/mg) or sPLA₂ (40-1,500 nmol/min/mg). Nevertheless, the described enzyme plays an important role in metabolic processes because RBCs have the portion of up to 99% of the blood cell mass.

The activity of inventive enzyme is not inhibited by DTT (dithiothreitol) and mepacrine (sPLA₂ inhibitor) as is the cPLA₂ enzyme. The enzyme, however, is inhibited by AACOCF₃ (arachidonylfluoromethyl ketone) and activated by divalent metal cation such as Zn²⁺, Fe²⁺, Cu²⁺, Sr²⁺, Ba²⁺, Mn²⁺, Mg²⁺ and the like.

The described enzyme is highly expressed in murine fetal liver cells (MFL) where RBC precursor cells exist in abundance. Expression of the described enzyme, however, is not induced by EPO (erythropoietin), which is RBC hematopoietic factor that activates the expression of various gene in MFL cells. However, since the differentiation of RBC to MFL cell induced in proposition to the expression rate of the described enzyme, the described enzyme is correlated with RBC hematopoesis by a different pathway than EPO.

As described above, the described enzyme metabolizes similar substrates and shows a similar calcium-dependence and optimal pH range to conventional cPLA₂. The described enzyme also has a similar sensitivity to various chemical compounds or divalent metal cations as that demonstrated by the conventional cPLA₂ enzyme. However, there are several biochemical, immunological characteristics differences and the sensitivity to specific chemical compounds that distinguish the described enzyme from conventional cPLA₂. For the biochemical characteristics, the described enzyme shows quite different phenomenon with cPLA₂ as a result of prosecuting various column chromatography. For the immunological characteristics, the described enzyme does not react with the antibodies against cPLA₂ or sPLA₂. For the sensitivity to the chemical compound, the described enzyme is not inhibited by methyl mercury, mercuric chloride or TP1 (2-(3,5-Di-tert-butyl-4-hydroxyphenyl)-3-chloro-1,4-naphthalene dione) known as conventional inhibitors of cPLA₂ or sPLA₂.

In accordance with another aspect of the present invention, there is also provided a process for preparing above-mentioned enzyme comprising the steps of subjecting the cytosolic fraction obtained by crushing RBC to butyl-Toyopearl hydrophobic column, first phenyl-5PW hydrophobic HPLC column, DEAE-5PW HPLC column, Sephacryl S-300 gel filtration column, a 2^{nd} phenyl-5PW hydrophobic HPLC, Superose 12 gel filtration FPLC column and Mono Q FPLC column chromatography consecutively.

In accordance with another aspect of the present invention, there is also provided an rPLA₂ antibody effectively reacting with above-mentioned rPLA₂ enzyme. The antibody can bind to described bovine enzyme as well as 42 kDa protein isolated from human RBC cytosol, however, the antibody does not react with cPLA₂ or sPLA₂. The antibody can be prepared by mixing the instant enzyme with an equivalent amount of adjuvant, injecting the mixture onto a mouse and collecting the serum from the mouse. For example, the active lysate of rPLA₂ enzyme obtained from a bovine source is concentrated to a concentration of 25 μg per 0.25 ml. The concentrated protein solution is mixed with the equal amount of adjuvant and then the mixture is injected into mouse at the interval ranging one week to three weeks, from three to four times, preferably four times. The immunized mouse is sacrificed and the serum was obtained.

In accordance with another aspect of the present invention, there is also provided an pharmaceutical composition comprising a therapeutically effective amount of EA4 (7-chloro-6-[4-(diethylamine)phenyl]-5,8-quinolinedione) compound and a pharmaceutically acceptable carrier. This composition has utility in treating diseases caused by disorder of hematopoietic system related to rPLA₂ enzyme.

In accordance with another aspect of the present invention, there is also provided a use of EA4 (7-chloro-6-[4-(diethylamine)phenyl]-5,8-quinolinedione) compound for inhibiting rPLA₂ enzyme. EA4 can inhibit the activity of rPLA₂ competitively at the inhibition constant (Ki) of 130 μM and inhibits the release of Ca²⁺-dependent arachidonic acid in bovine RBC. Moreover, EA4 inhibits the activity of cPLA₂, which is different from conventional PLA₂ inhibitors that show activity against only one species of PLA₂.

Accordingly, the described rPLA₂ enzyme, antibodies with specific activity against the enzyme, and the EA4 compound can be useful to treat or prevent rPLA₂ related diseases and to study the physiological phenomena related to RBC such as hemostasis, thrombosis and RBC hematopoiesis, and others.

For example, when the expression, function and regulation mechanism of rPLA₂ enzyme are found out, the metabolic pathway of metabolites produced by rPLA₂ and the origin of the diseases related to rPLA₂ are found out, they can help diagnosing, preventing and treating various diseases related to rPLA₂ by inducing a mutuation in rPLA₂ resulting in the reduction or inactivation of activity.

The described rPLA₂ enzyme can be used for the preparation of a pharmaceutical composition for the treatment of diseases caused by arachidonic acids. The pharmaceutically acceptable salt of each of the compounds may be a salt of an alkali metal, such as sodium and potassium, an alkali earth metal, such as magnesium and calcium, or ammonia or an organic base, such as, TEA, pyridine and picoline.

The described pharmaceutical formulation may be prepared in accordance with conventional procedures. For example, in preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulation may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, or diluents are lactose, dextrose, sucrose, sorbitol, mannitol, starches, gum acacia, alginates, gelatin, calcium phosphate, calcium silicate, cellulose, methylcellulose, microcrystalline cellulose, polyvinylpyrrolidone, water, methylhydroxybenzoates, propylhydroxybenzoates, talc, magnesium stearate and mineral oil. The formulation may additionally include fillers, anti-agglutinating agents, lubricating agents, wetting agents, flavoring agents, emulsifiers, preservatives and the like. The composition of the invention may be formulated so as to provide a quick, sustained or delayed release of the active ingredient after it is administrated to a patient, by employing any one of the procedures well known in the art.

The pharmaceutical formulation of the present invention can be administered via various routes including oral, transdermal, subcutaneous, intravenous and intramuscular introduction. For treating a human patient, a typical daily dose of the above-mentioned compounds isolated from Regina ferula may range from about 0.001 to 1 g/kg body weight, preferably 0.01 to 0.1 g/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The pharmaceutical composition comprising a specific antibody or inhibitor again rPLA₂ can be useful to the diagnosis, prevention and treatment of the diseases caused by an excessive activity of rPLA₂ such as thrombosis, arterioclerosis, cerebral infarction, chronic inflammation syndrome, immunological function disorder, cancer and the like.

The pharmaceutical composition comprising inventive antibody or inhibitor can be used alone or administrated in combination with treating methods such as surgery, radiotherapy, hormonal therapy, chemical therapy and biological response modulator.

### EXAMPLES

The following Examples are intended to further illustrate the present invention without limiting its scope.

### Example 1

The Preparation of rPLA₂ and the Identification of its Characteristics

1) Materials and Methods

Stearoyl-2-[1-¹⁴C]arachidonyl-sn-glycerol-3-phosphocholine (2-[1-¹⁴C]AA-GPC) (55.3 mCi/mmol), 1-palmitoyl-2-[1-¹⁴C]palmitoyl-sn-glycerol-3-phosphocholine (2-[1-¹⁴C]PA-GPC) (55.6 mCi/mmol), 1-palmitoyl-2-[1-¹⁴C]linoleoyl-sn-glycerol-3-phosphocholine (2-[1-¹⁴C]LA-GPC) (55.9 mCi/mmol), 1-acyl-2-[1-¹⁴C]arachidonyl-sn-glycerol-3-phosphoethanolamine (2-[1-¹⁴C]AA-GPE) (55.1 mCi/mmol) and [³H]arachidonic acid (AA) (204 Ci/mmol) ([³H]AA) were purchased from the radio-chemical center, Amersham Life Science Ltd. (Buckinghamshire, UK). 1-Stearoyl-2-arachidonyl-sn-glycerol-3-phosphocholine (2-AA-GPC), dithiothreitol (DTT), A23187, 3,3′-diaminobenzidine (DAB), methylcellulose, erythropoietin and Sepharose 4B-200 gel filtration column were purchased from Sigma Co. (St. Louis, Mo.). Anti-human secretory 14 kDa sPLA₂ antibody was purchased from Upstate Biotechnology, Inc. (Lake Placid, N.Y.). Goat anti-rabbit- and anti-mouse-alkaline phosphatase conjugates were purchased from Santa Cruz Biotechnology Inc., Santa Cruz, Calif. Group IV cytosolic PLA₂ (cPLA₂) was purified from porcine spleen and anti-cPLA₂ polyclonal antibody was generated as described above(Kim, D. K., and Bonventure, J. V., Biochem. J., 294, 261-270, 1993). Group II secretory PLA₂ (sPLA₂) was partially purified from bovine platelets as described above (Hara, S et al., J. biochem., 105, 395-399, 1989). Butyl-Toyopearl 650 M gel, preparative Phenyl-5PW, analytical Phenyl-5PW, DEAE-5PW HPLC columns were purchased from Tosoh Co. (Tokyo, Japan). Sephacryl S-300 gel filtration, Superose 12 gel filtration, PD-10 desalting (Sephadex G-25 M) and Mono Q FPLC columns, and Protein A-Sepharose CL-4B beads were purchased from Amersham Pharmacia Biotech (Uppsala, Sweden). Arachidonyl trifluoromethyl ketone (AACOCF₃) was obtained from Biomol (Plymouth Meeting, Pa.). Complete Freund's adjuvant and minimal essential medium (MEM) were obtained from Gibco BRL Life Technologies Inc (Grand Island, N.Y.). All other chemicals were of the highest purity or molecular biology grade available from commercial sources.

2) Identification of the rPLA₂ Activity in Human and Bovine RBCs.

To identify the rPLA₂ activity in human and bovine RBCs, the releasing activity of arachidonic acid was determined.

Human venous blood was collected in heparin (40 unit/ml) from some healthy volunteers among the Korean graduate students in our laboratory and bovine blood freshly collected in heparin (40 unit/ml) in a local slaughterhouse. After blood was centrifuged at 500×g for 20 min, the resulting supernatants of the platelet-rich plasma, the buffy coat and the leading edge of the packed RBCs were completely removed by aspiration. Sedimented RBCs, leukocytes, and platelets were re-suspended in a sterile buffer (50 mM Tris-HCl, pH 7.5, 1 mM EDTA, 0.12 M NaCl). This centrifugation and aspiration cycle was repeated six times taking care of removing leukocytes and platelets and the top 10% of the RBCs suspensions. Washed cell suspensions (10 ml) were subsequently depleted of residual leukocytes and platelets by filtration through a Sepharose 4B-200 column (20×2.5 cm) pre-equilibrated with sterile saline (0.9% w/v NaCl) as described above.

The filtered cell suspensions contained the following numbers of blood cells: for human blood; <3×10⁵ platelets/ml, <2×10⁴ leukocytes/ml, and 4-5×10⁹ RBCs/ml; for bovine blood; <4×10⁵ platelets/ml, <3×10⁴ leukocytes/ml, and 3-5×10⁹ RBCs/ml. Differential cell counts were measured with a Coulter counter (Becton Dickinson UK, Oxford, UK).

The Sepharose 4B-200 column-purified RBCs suspensions (approximately 1×10⁹ cells/ml) were twice washed with serum-free MEM containing 1 mg/ml of fatty acid-free bovine serum albumin (BSA) and labeled for 1 hr with 1.5 mCi [³H]AA (1 mCi/0.1 ml ethanol)/ml of the same medium. Murine L929 cells (1-2×10⁶ cells/ml) were labeled for 6 hrs with 1.5 mCi [³H]AA (1 mCi/0.1 ml ethanol)/ml of the same medium. Thereafter, cells were washed three times to remove all unincorporated [³H]AA. The labeled cells were incubated in MEM containing 1 mg/ml BSA as a trap for the released [³H]AA and then stimulated with vehicle (0.1 μl ethanol/ml medium) or 2 μM of calcium ionophore A23187 at 37° C. And a control group is treated with vehicle only without A23187 compound.

For analysis of [³H] AA release, at each time of 0, 10, 30, 60 and 90 min after reaction between the cell and A23187, the RBCs were centrifuged as above, and each aliquot (200 μl) of the supernatants for the RBCs and each aliquot (100 μl ) of the conditioned media for the L929 cells was transferred to 2.5 ml of the scintillation counting solution and counted for radioactivity with a Packard Tri-carb liquid β-scintillation counter (Packard Instrument Co., Meriden, Conn.).

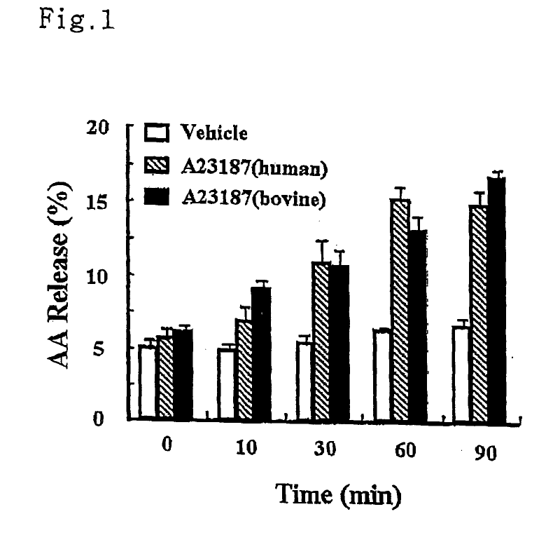

Since the higher value of radioactivity shows that arachidonic acid labeled with radioactive isotope is released more, above experiment was repeated to assess statistical data shown in FIG. 1.

As shown in FIG. 1, a calcium ionophore A23187 released [³H]AA from the purified human and bovine RBCs in a time-dependent manner. The release of [³H]AA in these cells were relatively rapid as significantly observed at 10 min and gradually increased up to 60 min, which means that there exists the calcium ion dependent rPLA₂ activity in human and bovine RBCs.

To identify to which class type of the rPLA₂ in human and bovine RBCs is belonged, the substrate specificity of rPLA₂ in human and bovine RBCs was determined.

The total incorporated [³H]AA into the RBCs was determined by centrifuging the RBC suspensions at 10,000×g for 1 min immediately and 1 hr after addition of [³H]AA, respectively, and measuring the radioactivity of each aliquot of the supernatants. The total incorporated [³H]AA into L929 cells was measured by counting the radioactivity of an aliquot (50 μl) of the cell lysates obtained after washing the cells three times with 10 ml of PBS and then adding 1 ml of 0.5 N NaOH solution. Then, 2-[1-¹⁴C]AA-GPC (55.3 mCi/mmol), 2-[1-¹⁴C]LA-GPC (1-palmitoyl-2-[1-¹⁴C]linoleoyl-sn-glycerol-3-phosphocholine, 55.9 mCi/mmol), 2-[1-¹⁴C]PA-GPC (1-palmitoyl-2-[1-¹⁴C]palmitoyl-sn-glycerol-3-phosphocholine, 55.6 mCi/mmol) and 2-[1-¹⁴C]AA-GPE (1-acyl-2-[1-¹⁴C]arachidonyl-sn-glycerol-3-phosphoethanolamine, 55.1 mCi/mmol) was used as substrate respectively.

As result of above experiment, a Ca²⁺-dependent PLA₂ activity, which preferred 2-[1-¹⁴C]AA-GPC to 2-[1-¹⁴C]LA-GPC and 2-[1-¹⁴C]PA-GPC by 8.5- and 25.2-folds, respectively, was detected in the cytosolic fractions and hydrolyzed preferentially 2-[1-¹⁴C]AA-GPE to 2-[1-¹⁴C]AA-GPC by 1.7-fold.

This substrate specificity for the RBC form of PLA₂ from the cytosolic fractions suggests that this enzyme may be similar to a group IV cPLA₂.

3) Purification of rPLA₂ from Bovine RBCs

To isolate rPLA₂ from bovine RBCs cytosol, various kinds of column chromatographic methods were subjected to purify the rPLA₂ protein.

Additionally, to compare the biochemical characteristics between rPLA₂ in bovine RBCs and common group IV cPLA₂, as a control group, the porcine spleen tissue abundant in cPLA₂ was subject to purification process in a same manner.

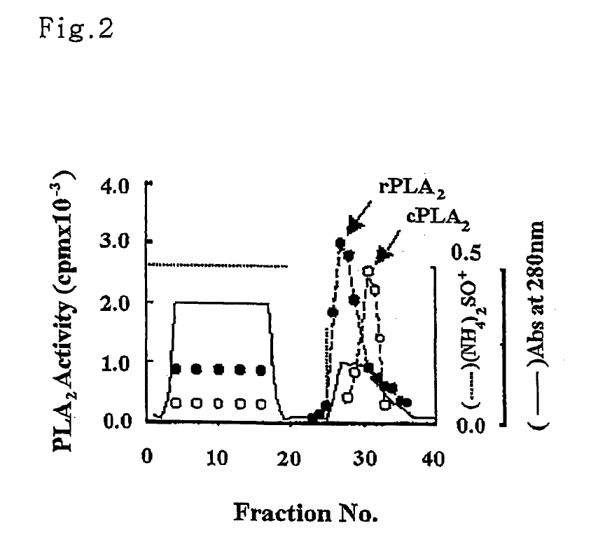

The packed RBCs were prepared from bovine blood (4 liters) as described above and re-suspended in buffer A (50 mM Tris-HCl, pH 7.5, 1 mM EDTA, 10 mM 2-mercaptoethanol) containing 1 μg/ml leupeptin, 5 mg/ml aprotinin, 1 mM DTT and 1 mM phenylmethylsulfonyl fluoride and used as the enzyme source for purification of PLA₂. Firstly, to obtain cytosolic and membrane fractions of bovine RBCs, the resuspended packed cells were homogenized by sonicating in an ice bath at 40 W output and 40% duty cycle for 20 seconds with a sonicator (Sonics & Materials inc., Danbury, Conn.). The debris and unlysed cells were removed by centrifuging the homogenates at 3,000×g at 4° C. for 30 min. After the supernatants were again centrifuged at 100,000×g at 4° C. for 2 hours, the resulting supernatants and pellets were obtained as the cytosolic and membrane fractions, respectively. For the first step, the cytosolic fractions were adjusted to 0.5 M (NH₄)₂SO₄, stirred at 4° C. for 5 min and loaded onto a Butyl-Toyopearl hydrophobic column (15.0 cm×5.0 cm) pre-equilibrated with buffer A containing 0.5 M (NH₄)₂SO₄ at a flow rate of 20 ml/min. After washing with buffer A until no protein was eluted, the column-binding proteins were eluted at a flow rate of 20 ml/min with a stepwise gradient of distilled water. The analysis data for determining the activities of each eluted fractions was shown in FIG. 2.

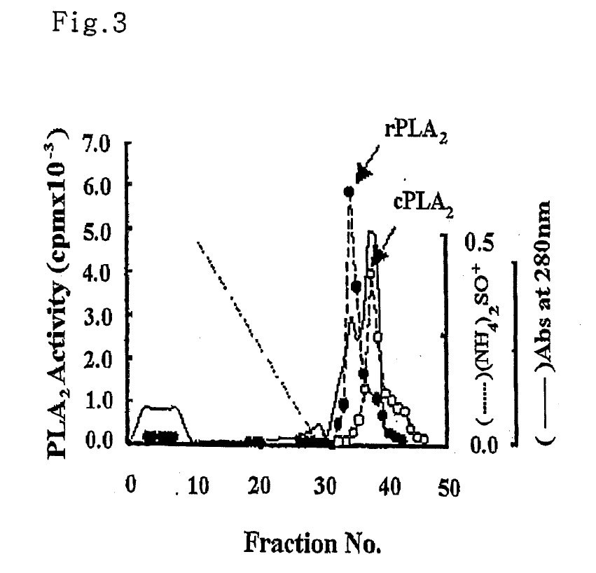

Next, a pool of the active fractions was adjusted to 0.5 M (NH₄)₂SO₄ and then loaded onto a preparative Phenyl-5PW hydrophobic HPLC column (21.3 mm×15 cm) pre-equilibrated with buffer A containing 0.5 M (NH₄)₂SO₄ at a flow rate of 5.0 ml/min. The column-binding proteins were eluted at a flow rate of a 100 ml linear gradient of 0.5−0.0 M (NH₄)₂SO₄, and 5 ml fractions were collected. The analysis data for determining the activities of each eluted fractions was shown in FIG. 3.

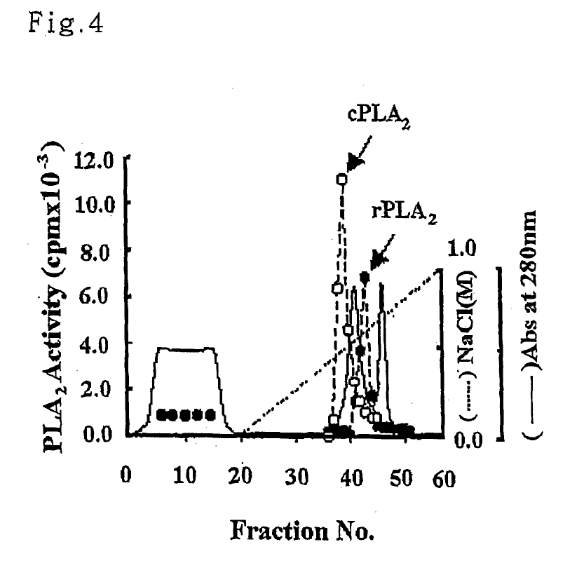

The active fractions were pooled and loaded onto a DEAE-5PW HPLC column (7.5 mm×7.5 cm) pre-equilibrated with buffer A at a flow rate of 1.0 ml/min. Proteins bound to the column were eluted with a 20-ml linear gradient of 0.0-1.0 M NaCl, and 1 ml fractions were collected. The analysis data for determining the activities of each eluted fractions was shown in FIG. 4.

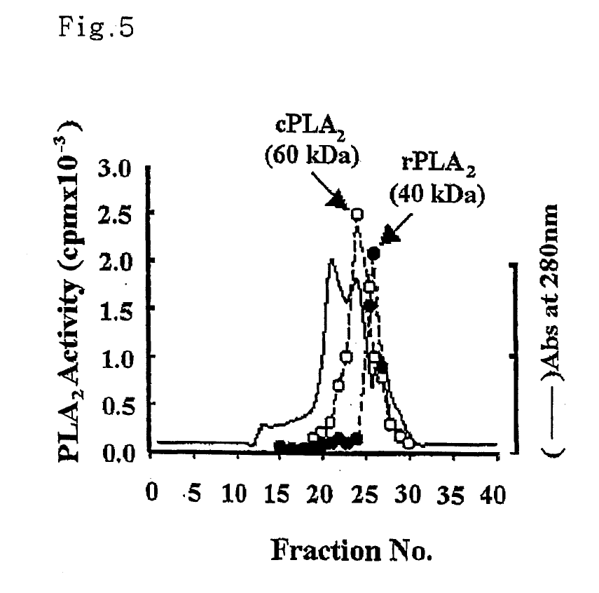

The active fractions from the DEAE-5PW column were pooled and injected onto a Sephacryl S-300 gel filtration column (30 mm×60 cm) pre-equilibrated with buffer A containing 0.1 M NaCl. The column was eluted with the same buffer at a flow rate of 1 ml/min. The active pool was continuously adjusted to 0.5 M (NH₄)₂SO₄ and then loaded onto an analytical Phenyl-5PW hydrophobic HPLC column (7.5 mm×7.5 cm) pre-equilibrated with buffer A containing 0.5 M (NH₄)₂SO₄ at a flow rate of 1.0 ml/min. The column-binding proteins were eluted at a flow rate of 1 with a 20 ml linear gradient of 0.5−0.0 M (NH₄)₂SO₄. The fractions of the major peak activity eluted were pooled and used for further purification. The active pool was concentrated into approximately 250 μl using a Centricon 10 (Amicon Co., Beverly, Mass.) and injected onto a Superose 12 gel filtration FPLC column (10 mm×30 cm) pre-equilibrated with buffer A containing 0.1 M NaCl. The column was eluted with the same buffer at a flow rate of 0.5 ml/min. 0.5 ml fractions were collected. The analysis data for determining the activities of each eluted fractions was shown in FIG. 5.

Finally, this active fractions were loaded onto a Mono Q FPLC column (5.0 mm×5.0 cm) pre-equilibrated with buffer A adjusted to pH 8.0 at a flow rate of 1.0 ml/min. Proteins bound to the column were eluted with a 20 ml linear gradient of 0.0-1.0 M NaCl, and 1 ml fractions were collected. To monitor the amount of protein during purification of rPLA₂, the absorbance at 280 nm (A₂₈₀) was measured by an UV detector. Protein concentration of each sample was measured with Bradford reagents (Bio-Rad, Hercules, Calif.) using BSA as a standard.

As shown in FIGS. 2 to 5, the bovine RBCs cytosolic rPLA₂ was eluted at different fractions from all of the columns utilized differently from cPLA₂ and in particular, the RBCs rPLA₂ migrated as a molecular mass of about 40 kDa comparing with the cPLA₂ having a molecular mass of about 60 kDa.

Therefore, it is confirmed that the biochemical characteristic of bovine RBCs cytosolic rPLA₂ is different from that of known cPLA₂.

The summarized result of above column chromatographic experiment is shown in Table 1.

TABLE 1
Summary of purification of rPLA₂ from bovine RBCs

| | | | | Specific | |
|---|---|---|---|---|---|
| | Total | Total | | activity | |
| Purification | protein | activity | Yield | (pmol/min/mg | |
| step | (mg) | (pmol/min) | (%) | protein) | Fold |
| S100 | 46,000 | 18,400 | 100.0 | 0.4 | 1 |
| Butyl- | 700 | 7,280 | 39.6 | 10.4 | 26 |
| Toyopearl | | | | | |
| Phenyl- | 37.50 | 4,095 | 22.3 | 109.2 | 273 |
| 5PW(I) | | | | | |
| DEAE-5PW | 7.00 | 1,873 | 10.2 | 267.6 | 669 |
| Sephacryl | 1.70 | 1,804 | 9.8 | 1061.2 | 2,653 |
| S-300 | | | | | |
| Phenyl- | 0.52 | 656 | 3.6 | 1,261.6 | 3,154 |
| 5PW(II) | | | | | |
| Superose 12 | 0.25 | 412 | 2.2 | 1,648.0 | 4,120 |
| Mono Q | 0.06 | 336 | 1.8 | 5,600.0 | 14,000 |

As can be seen from Table 1, two hydrophobic columns as initial steps typically resulted in a 273-fold purification and 22.3% yield of bovine RBC cytosolic PLA₂. A Superose 12 gel filtration FPLC column resulted in a 1.3-fold purification with an efficient yield of 62% and Mono Q anion-exchange FPLC column resulted in a 3.4-fold purification with a high yield of 81%.

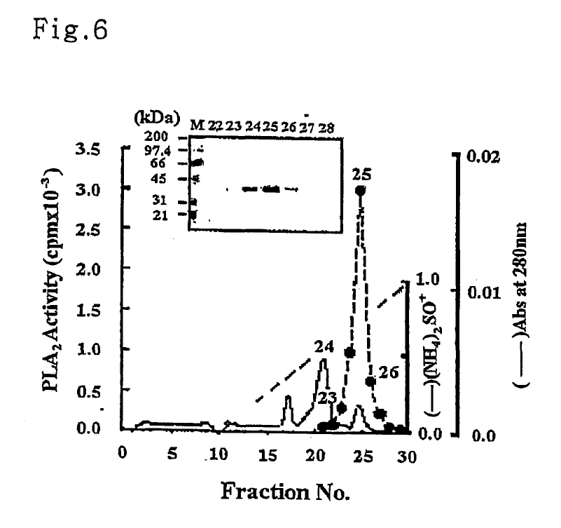

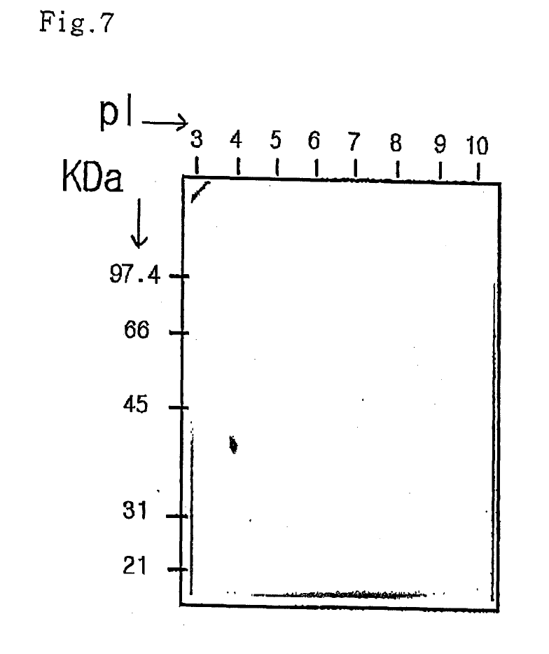

To assess the purity, a portion of each fraction was analyzed on one-dimensional and two-dimensional SDS-PAGE gels, respectively. The results were shown FIGS. 6 and 7 respectively.

The relative PLA₂ activity from the final step paralleled the intensity of the 42 kDa band as a single protein band (See FIG. 6, inset), and a single spot was observed in a two-dimensional SDS-PAGE (See FIG. 7), indicating that this 42 kDa band represents the RBC PLA₂, termed rPLA₂. MALDI-TOF mass spectrometric analysis of the single spot showed no apparent homology to any known protein.

Therefore, we confirmed that the purified rPLA₂ is novel protein through above experiments.

4) Immunochemical Study of rPLA₂

To identify the immunochemical characteristics of rPLA₂, following experiment was subjected.

Preparation of SDS-PAGE

One-dimensional denaturing SDS-PAGE was performed on 10% polyacrylamide gels according to Laemmli's procedure in a Bio-Rad Protean II electrophoresis system. Two-dimensional gel electrophoresis was performed according to O'Farrell method (O'Farrell, P. H., J. Biol. Chem., 250, 4007-4021, 1975) using the IPG-phor (Amersham Pharmacia Biotech, Uppsala, Sweden) system according to the instructions of the manufacturer. The separated proteins were stained with a PlusOne silver staining kit (Pharmacia Biotech Inc. Piscataway, N.J.).

Immunochemical Study

The cPLA₂ isolated from porcine spleen (PS-cPLA₂) and sPLA₂ isolated from bovine platelet (BP-sPLA₂) were adopted as comparative groups and the anti-serums for each enzyme were prepared to be subjected to immunoprecipitation process as follows.

For immunoprecipitation study, pre-immune serum (50 μl) and anti-42 kDa protein antiserum (50 μl) were mixed with packed Protein A-Sepharose CL-4B beads (bed volume, 25 μl), respectively, and incubated overnight at 4° C. The beads were then washed six times with 1.0 ml of buffer B (20 mM Tris-HCl, pH 7.5, 1 mM EDTA, 2.0% (w/v) BSA) and incubated with an active pool (protein 8.2 μg) from the Superose 12 column for the indicated times at 4° C. with constant shaking. Then, the beads were pelleted by centrifuging at 1,300×g at 4° C. for 1 min, and each aliquot of the resulting supernatants was assayed for PLA₂ activity. The pellets were washed six times with buffer B containing 0.1% Tween 20 and 0.5 M NaCl.

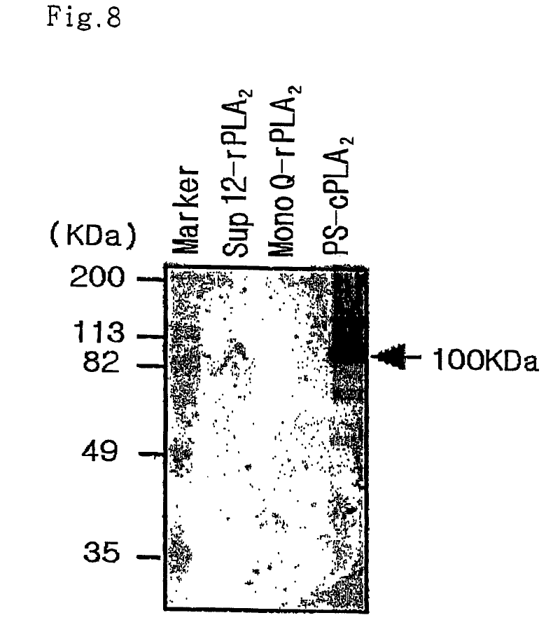

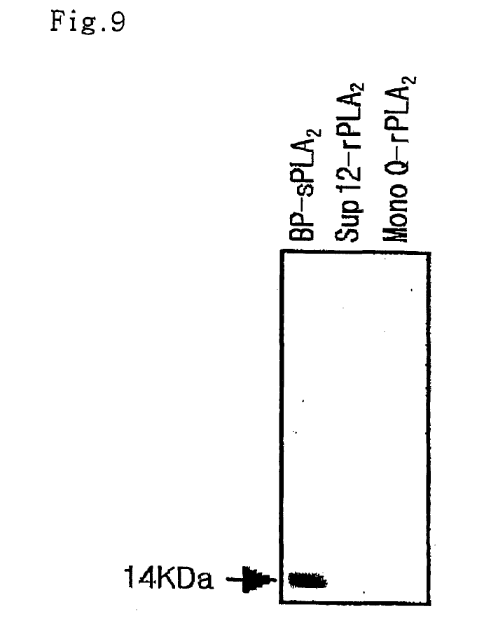

The washed bead was subjected to electrophoresis analysis using 10% polyacrylamide gel and then the proteins isolated from the gel were transferred to nitrocellulose membrane (Hybond™ ECL™ nitrocellulose membrane, Amersham Pharmacia UK Ltd.). The membrane was reacted with primary antibody wherein the dilution ratio of the antiserum was 1:2000. After primary antibody reaction, the 1:2,500 dilution of goat anti-rabbit or anti-mouse-alkaline phosphatase conjugate was adopted as secondary antibody. After antibody reaction, the result was identified using a preformulated substrate kit (1-Step™ NBT/BCIP; Pierce Co., Rockford, Ill.) shown in FIGS. 8 and 9.

As can be seen in FIGS. 8 and 9, rPLA₂ did not react with anti-spleen cPLA₂ (See FIG. 8) and anti-sPLA₂ antisera (See FIG. 9) in immunoblotting analysis, therefore, we confirmed that the immunological characteristics of rPLA₂ are quietly different from those of other types of PLA₂s.

5) Enzymatic Characteristics of rPLA₂

To identify the characteristics of rPLA₂, the reaction velocity with substrate, specificity for substrate, calcium ion dependence, pH dependence, PLA₂ inhibitor and the sensitivity to divalent metal ion were analyzed as follows.

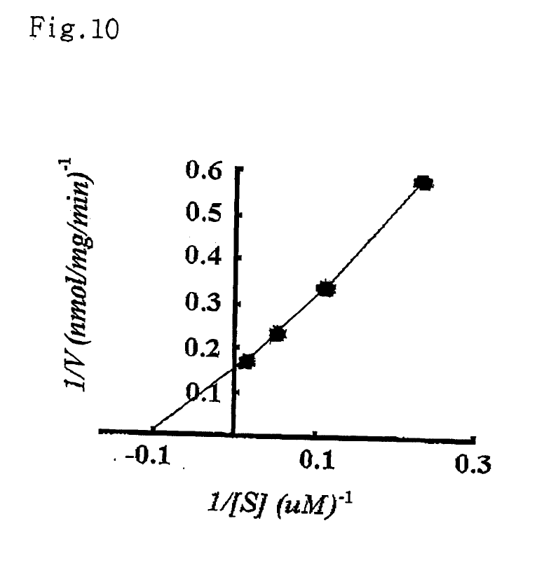

The active pool from Mono Q column was desalted by using PD-10 desalting column equalized with 10 ml of Tris (pH 7.5) buffer solution and was reacted with various concentrations of 2-[1-¹⁴C]-AA-GPC. The result shows that the apparent Km value was 13.9 μM and the Vmax value was 7.4 nmol/min/mg of protein with 2-[1-¹⁴C]AA-GPC (See FIG. 10).

To verify the substrate specificity of rPLA₂, the active pool from Mono Q column was reacted with various types of phospholipids containing AA together with cPLA₂ as a comparative group. Each velocity of aliquot of these PLA₂ enzymes was adjusted to 0.18-0.21 nmol/10 min per 45 μM of 2-[1-¹⁴C]-AA-GPC and 0.9 μM each substrate was used.

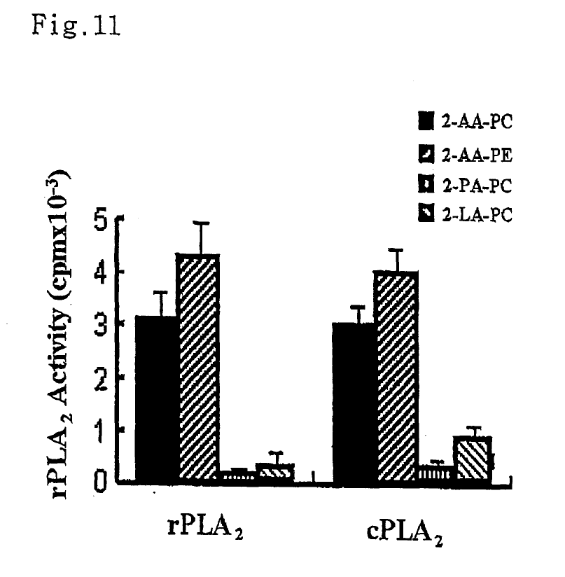

The result shows that rPLA₂ has the high selectivity for phospholipids containing AA at the sn-2 position like cPLA₂ (See FIG. 11).

To verify the calcium ion effect on the activity of rPLA₂, after various concentrations of calcium ion were added to the active pool from Mono Q column and reacted at 37° C., for 5 min, 2-[1-¹⁴C]-AA-GPC as a substrate was added and reacted for further 10 min. The comparative group, cPLA₂, was subjected to in a same procedure.

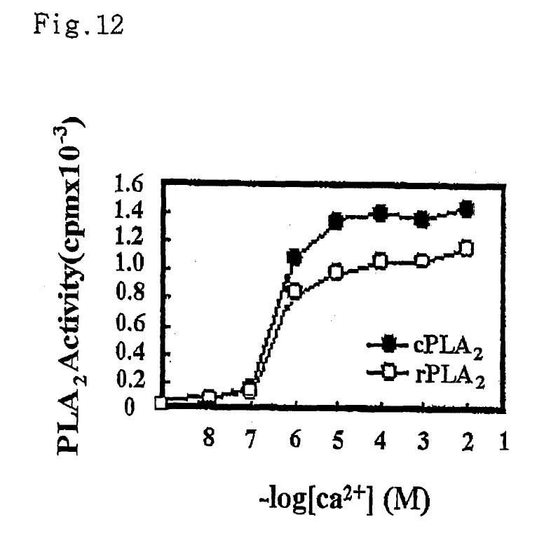

The result reveals that rPLA₂ shows calcium ion dependent activity profile similar to cPLA₂ (See FIG. 12).

To verify the pH effect on the activity of rPLA₂, the active pool from Mono Q column was titered in various pH and reacted 2-[1-¹⁴C]-AA-GPC as a substrate was added and reacted for 10 min. The comparative group, cPLA₂, was subjected to in a same procedure.

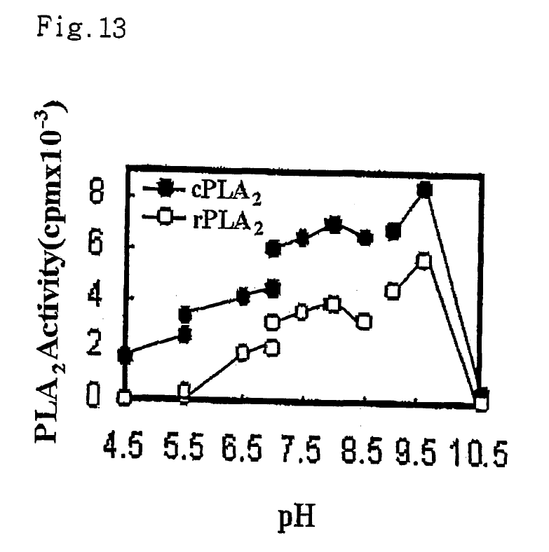

The result reveals that rPLA₂ shows maximum activity at the pH ranging from 9.5 to 10 (See FIG. 13).

To verify the effect of known inhibitors on the activity of rPLA₂, the various concentrations of inhibitors, i.e., DTT (dithiothtreitol), AACOCF3 (arachidonylfluoromethyl ketone) known as the cPLA₂ inhibitor, mepacrine and methyl mercury, mercuric chloride known as the sPLA₂ inhibitors were added to active pool from Mono Q column and reacted 2-[1-¹⁴C]-AA-GPC as a substrate for 10 min. The comparative groups, cPLA₂ and sPLA₂, were subjected to in a same procedure.

The result reveals that rPLA₂ shows maximum activity at the pH ranging from 9.5 to 10 (See FIG. 13).

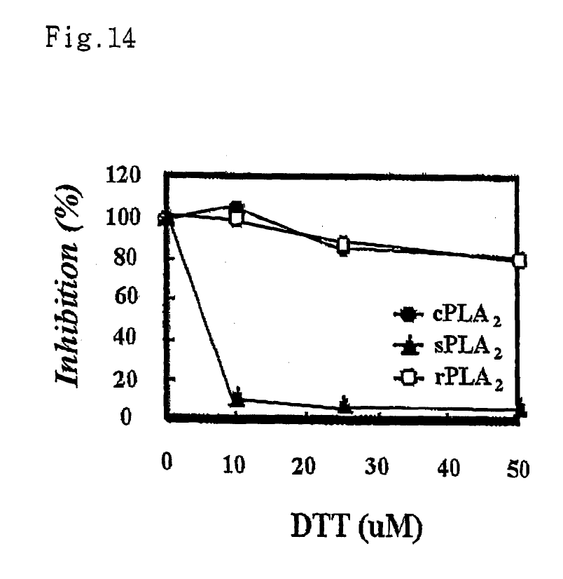

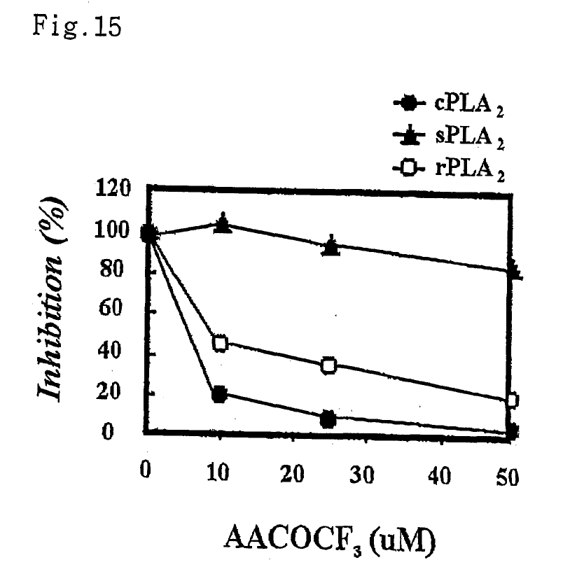

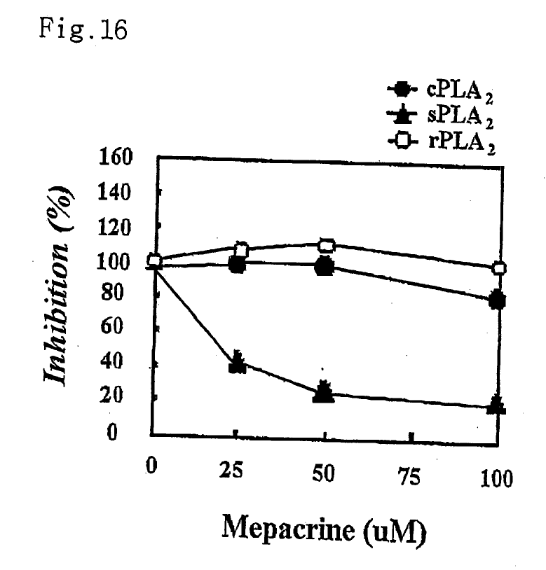

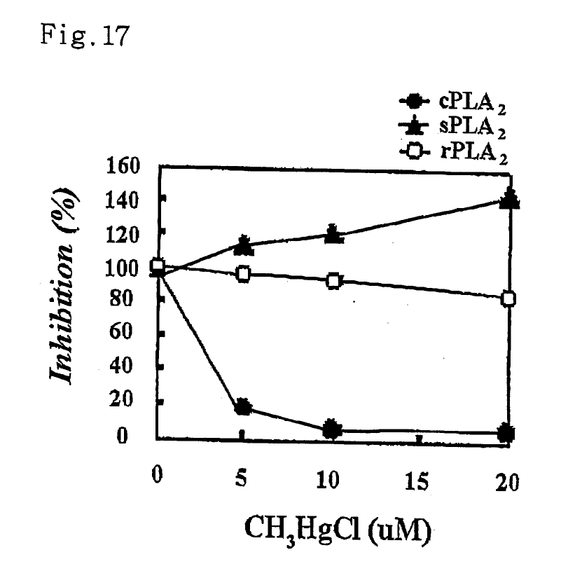

As shown in FIGS. 14, 15 and 16, rPLA₂ shows similar activity to cPLA₂, however, rPLA₂ showed less effective activity than cPLA₂ when treated with methyl mercury (See FIG. 17) and showed different profile from cPLA₂ when treated with mercury chloride.

To verify the effect of divalent metal ions on enzyme activity, to rPLA₂ activity, various concentrations of divalent metal i.e., Zn²⁺, Fe²⁺, Cu²⁺, Sr²⁺, Ba²⁺, Mn²⁺ and Mg²⁺ were added to active pool from Mono Q column and reacted 2-[1-¹⁴C]-AA-GPC as a substrate for further 10 min. The comparative groups, cPLA₂ and sPLA₂, were subjected to in a same procedure.

The result reveals that activity of rPLA₂ is similar to that of cPLA₂.

Therefore, rPLA₂ shows similarities to cPLA₂ in substrate specificity, calcium ion dependence, pH dependence, PLA₂ inhibitor and the sensitivity to divalent metal ion, whereas shows a little different response for enzyme inhibitors.

### Example 2

The Preparation of rPLA2 Antibody for and the Identification of rPLA₂ Function using Them.

1) The Preparation of rPLA₂ Antibody

To prepare mouse anti-42 kDa protein polyclonal antibody, the active pool obtained from the Mono Q column was concentrated using a Centri-Prep (Amicon Co., Beverly, Mass.) by ˜5-fold and an aliquot (˜25 μg of protein in 0.25 ml) was mixed with the same volume of complete Freund's adjuvant and injected into a BALB/c mouse via intraperitoneal route. After boosting four times at a 3-week interval, the immunized mouse was sacrificed and the serum was obtained, and it had been used as antiserum containing the antibody for 42 kDa protein for further experiments (thereinafter, 42 kDa Ab)

2) The Identification of the rPLA₂ Activity for AA Release using rPLA₂ Ab

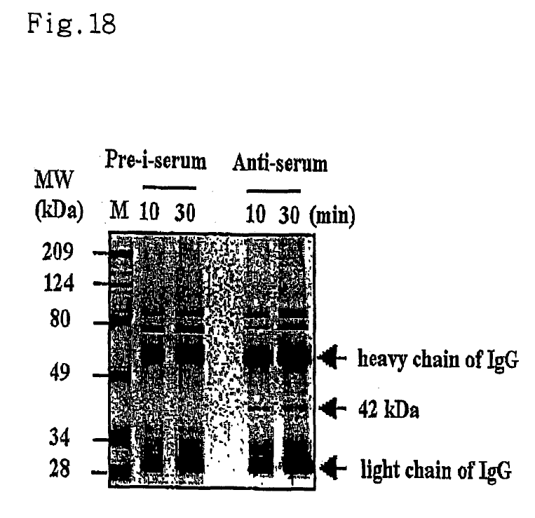

For immunoprecipitation study, pre-immune serum (50 μl ) and anti-42 kDa protein antiserum (50 μl) were mixed with packed Protein A-Sepharose CL-4B beads (bed volume, 25 μl), respectively, and incubated overnight at 4° C. The beads were then washed six times with 1.0 ml of buffer B (20 mM Tris-HCl, pH 7.5, 1 mM EDTA, 2.0% (w/v) BSA) and incubated with an active pool (protein 8.2 μg) from the Superose 12 column for the indicated times at 4° C. with constant shaking. Then, the beads were pelleted by centrifuging at 1,300×g at 4° C. for 1 min, and each aliquot of the resulting supernatants was assayed for PLA₂ activity. The pellets were washed six times with buffer B containing 0.1% Tween 20 and 0.5 M NaCl, separated on 10% SDS-PAGE and visualized by a silver staining kit. The result was shown in FIG. 18.

As shown in FIG. 18, 42 kDa protein band was detected when reacted with the active pool of Superose 12 column, the density of band was increased with the reaction time of bead and antiserum, whereas the control group did not show any band excluding an IgG band commonly showed at immunoprecipitation. Therefore, it is confirmed that the antibody prepared by above example directly react with rPLA₂.

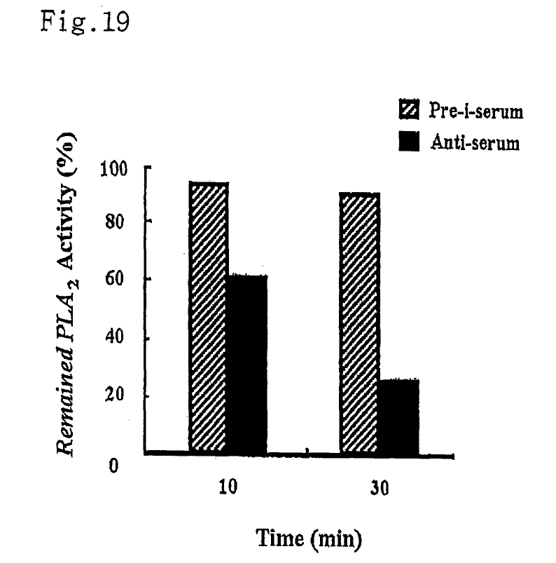

To measure the functions of rPLA₂ using inventive antibody, the supernatant obtained from above procedure was adopted to analyze the activity. PLA₂ activity was measured in an assay system (100 μl) of 75 mM Tris-HCl (pH 7.5) containing 45.0 mM of 2-[1-¹⁴C]AA-GPC (110,000 cpm/4.5 nmol) mixed with 2-[1-¹⁴C]AA-GPC, 4% glycerol, 5 mM CaCl₂ and 0.2 % BSA. The result was showed in FIG. 19.

As shown in FIG. 19, when 42 kDa protein band was reacted with the active pool of Superose 12 column, the density of band was remarkably decreased with the reaction time of bead and antiserum, whereas the control group did not show remarkable effect. Therefore, it is confirmed that rPLA₂ has PLA₂ activity considering the antibody prepared by above example reduced PLA₂ activity.

To confirm the above result repeatedly, RBC protein pool obtained from the purification procedure subjected by first phenyl-5PW column chromatography and the bead were combined and then those were separated into two parts, the precipitates and supernatant.

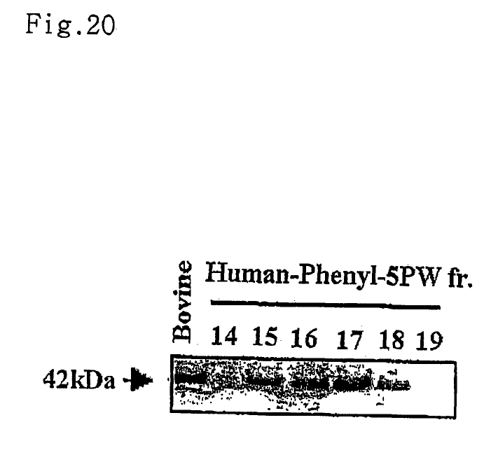

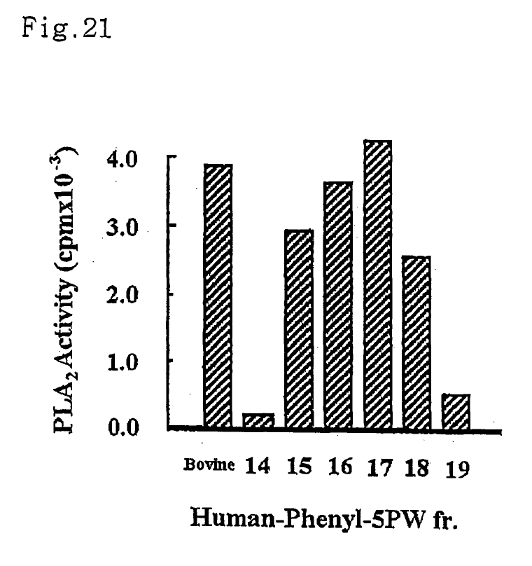

The immunoprecipitation method using above supernatant and precipitate were subjected according to the same method with the immunochemical study described in Example 1-(3) wherein the first antibody was used with the dilution solution (1:5000) of 42 kDa Ab. The results were shown in FIGS. 20 and 21 respectively.

As shown in FIG. 20, 42 kDa protein band was detected in the range of No. 15 to 18 fractions and the density was highest in No. 17 fraction. This result is consistent with the result in FIG. 21 showed that No. 17 fraction had highest rPLA₂ activity. The density of 42 kDa protein band was in proportion to the PLA₂ activity.

Accordingly, we confirmed that 42 kDa protein was bovine RBC PLA₂.

3) The Identification of the rPLA₂ Expression using rPLA₂ Ab and the Correlation with EPO

The rPLA₂ expression was verified in several cells and tissues with rPLA₂ Ab and whether the rPLA₂ expression can be induced by EPO or not was also verified.

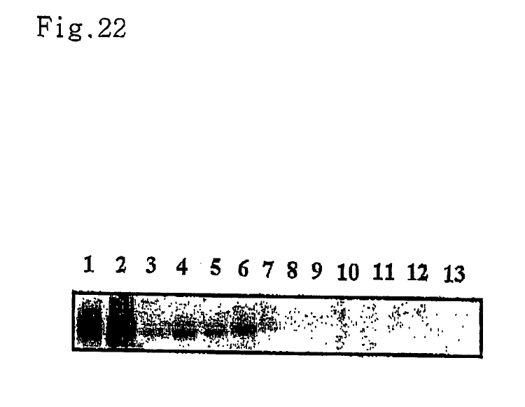

An rPLA₂ purified from phenyl-5PW column, rPLA₂ from Mono Q column and MDCK cell (lane No. 1, 2 and 3 each in FIG. 22), various units (0U, 0.2U and 0.5U) of EPO (lane No. 4, 5 and 6 each FIG. 22) for MFL cell and L929 cells, U937 cells, brain, kidney, lung, liver and spleen tissue of rat (lane No. 7, 8, 9, 10, 11, 12 and 13 each in FIG. 22) were prepared and treated in the experiment together.

Each cell was incubated for 1-2 weeks at 37° C. maintaining the number of the cells (2-3×10⁶ cells/ml) and each tissue was prepared by scarifying SD male rat.

Each cell and tissue were resuspended with Solution A containing 0.12M NaCl and then homogenized by sonicating at 4-watt output and 40% duty cycle for 20s with a sonicator and the homogenates were centrifuged at 100000×g at 4° C. for 1 hour.

Each 50 μg of protein was adopted and subjected to undergoing immunoprecipitation with 42 kDa. The result is shown in FIG. 22.

As shown in FIG. 22, the band density for 42 kDa protein did not show significant change although the treatment amount at EPO was increased.

Therefore, the result suggests that rPLA₂ expression is not induced by EPO.

4) The Effect of rPLA₂ on RBC Hematopoiesis using rPLA₂ Ab

To verify the effect of rPLA₂ on RBC hematopoiesis, we confirmed the correlation between the expression of rPLA₂ and pseudoperoxidase activity in hemoglobinized cell. The pseudoperoxidase was the enzyme publicly used as a marker for erythroid cell.

To confirm the change of pseudoperoxidase activity in hemoglobinized cell, MFL cell was incubated as follows.

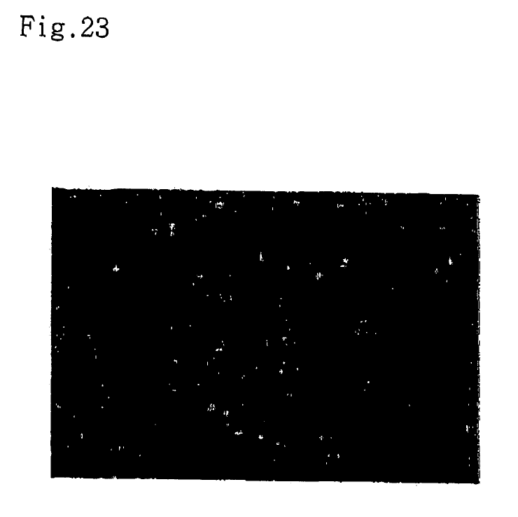

In order to obtain murine fetal liver (MFL) cells, adult male and female CD-1 mice (Dae Han Biolink Co., LTD., Eumsung-Gun, Chungbuk, Korea) underwent timed matings. At days 12-13 after mating, the female mice were killed while under ether anesthesia. According to the method of Mason-Garcia et al. (Mason-Garcia, M., et al., (1992) Am. J. Physiol. 262, C 1197-1203), the fetal livers were removed from the fetuses and gently teased free of the abdominal cavity. MFL cells were gently disaggregated by sequential passage through 18-, 21-, and 23-gauge hypodermic needles, washed twice in α-modified Eagle's minimum essential medium with glutamine (α-MEM; GIBCO, Grand Island. N.Y.), and resuspended in 5 ml α-MEM. Isolated murine fetal liver cells (1×10⁵/ml) were plated in a mixture (DAB-mixture) containing α-MEM, 0.8% methylcellulose, 20% fetal bovine serum, 10⁻⁴ M mercaptoethanol, 100 U/ml of penicillin, 100 μg/ml of streptomycin and 0.2 U/ml of highly purified human recombinant EPO (specific activity >160,000 U/mg protein). For DAB staining, 1 ml of the DAB-mixture was plated in each 10-×35-mm Petri dishes and incubated under a humidified atmosphere of 95% air and 5% CO₂. After 3 or 7 days, the dishes were stained for pseudoperoxidase with DAB and hydrogen peroxide and the result is shown in FIGS. 23 to 27.

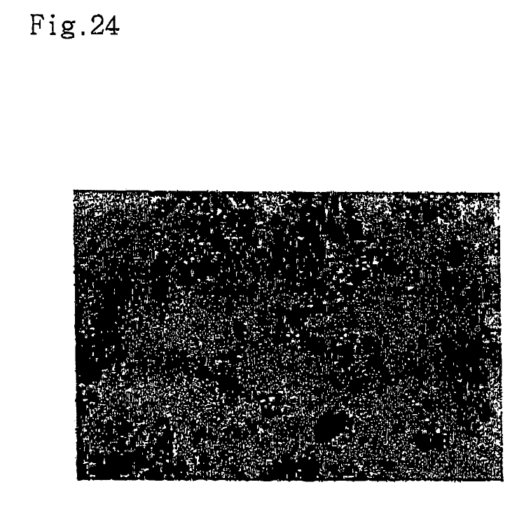

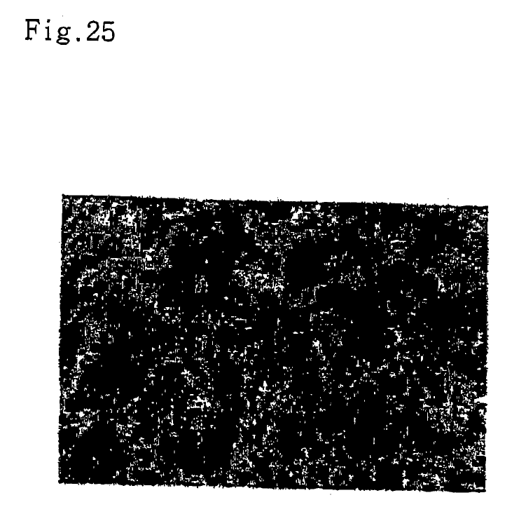

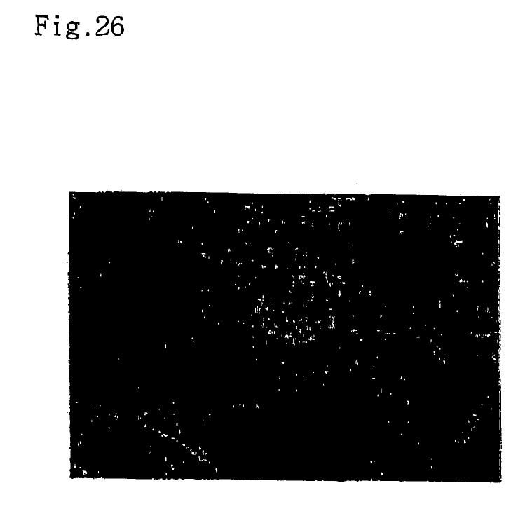

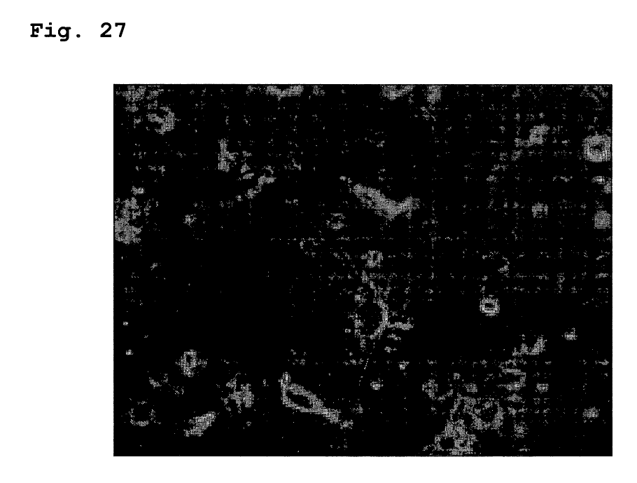

As shown in FIGS. 23 to 27, by 3 days culture, single cells were largely reduced (FIGS. 24 and 25) and instead DAB-positive colonies were found with majority as colony-forming unit erythroid (CFU-E), which consist of 10-20 cells with morphological appearance of basophilic erythroblasts and numerous mitotic figures. Most of the colonies were stained with brown color, which showed hemogloblinized. In contrast, by 7 days of culture, few erythroid colonies could be seen (FIGS. 26 and 27). There is no difference between EPO treated group (FIGS. 24, 26) and EPO untreated group (FIGS. 25, 27) in the cell hemoglobinization and EPO treated group reproduced more number of colonies (FIG. 25).

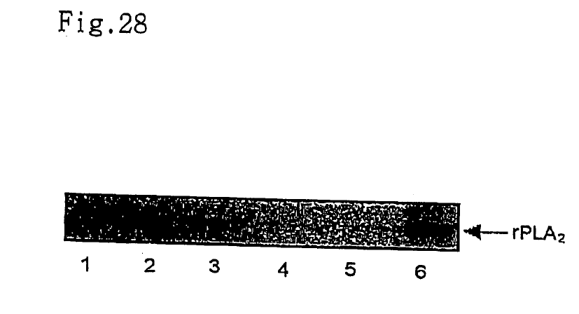

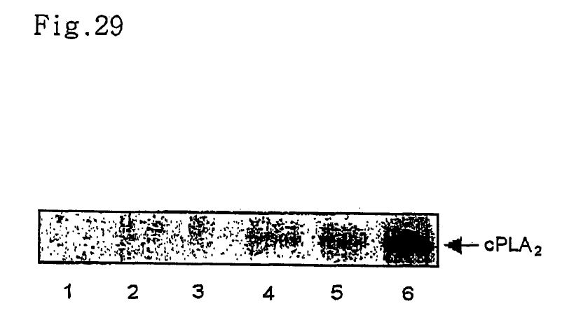

To verify whether above result is related to the expression of rPLA₂ or not, the protein obtained from above cultivated MFL cell and was subjected to immuno-precipitating method in same procedure described in Example 2-(2) using rPLA₂ Ab or cPLA₂ Ab. The result was shown in FIGS. 28 and 29 respectively. The protein obtained from the cell of FIG. 23 was loaded at lane No. 1 and the cell of FIGS. 24, 25, 26, 27 were loaded at Lane No. 2, 3, 4 and 5 respectively and positive control group was loaded at lane No. 6 to compare with the density of each protein band.

As shown in FIG. 28, the rPLA₂ of present invention was expressed by 3 days culture and was not expressed further by 7 days. This result is consistent with the cell hemoglobinization profile, which means the hematopoiesis of RBCs is induced with the activation of rPLA₂.

On the contrary, as shown in FIG. 29, the culture day of rPLA₂ increase, the expression increase. Therefore, we confirmed cPLA₂ do not effect on erythroid differentiation of MFL cell.

### Example 3

The Preparation of EA4 Compound, rPLA₂ Inhibitor and the Identification of rPLA₂ Function using Them.

1) The Preparation of EA4 Compound, rPLA₂ Inhibitor

An rPLA₂ inhibitor, EA4 was prepared by following method.

80 ml of acetic acid solution containing 6.28 mM 5,8-quinolinedione and cupric acetate monohydrate respectively was mixed with the 20 ml of acetic acid containing 6.28 mmol diethyl aniline. And the solution was stirred for 2 hrs at room temperature and left alone for one night. The mixture solution was filtered to obtain final precipitate. The chemical formula of EA4 compound is shown in chemical formula 1.

[Chemical Formula 1]

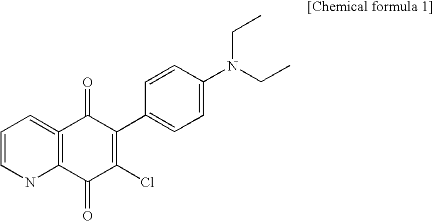

2) The Inhibitory Activity of EA4 Compound, rPLA₂ Inhibitor

To identify EA4 compound inhibits rPLA₂ enzyme, for control group, TP1, cPLA₂ inhibitor was EA4 was prepared.

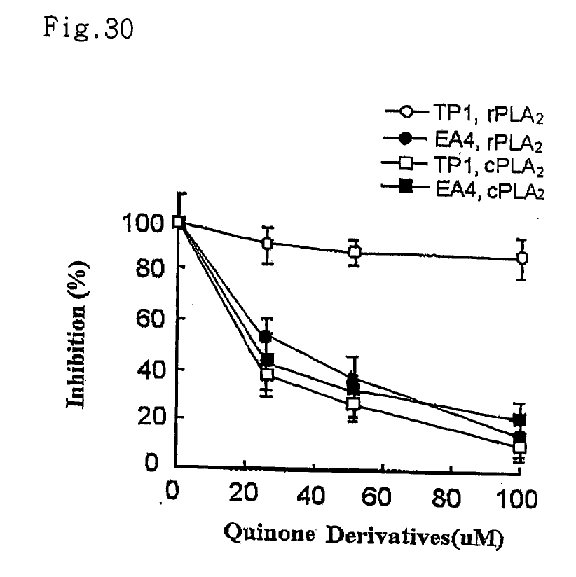

Prepared rPLA₂ and cPLA₂ and each inhibitor dissolved in 5 μl of DMSO solution was added and reacted for 10 min at 37° C. maintaining the temperature. 2-[1-¹⁴C]AA-PC as a substrate was added and reacted for 10 min at 37° C. maintaining the temperature. Inhibitory activities of EA4 and TP1 were estimated and showed the result in FIG. 30.

Shown in FIG. 30, EA4 inhibited both activities of rPLA₂ and cPLA₂, however TP1 inhibited that of cPLA₂ only.

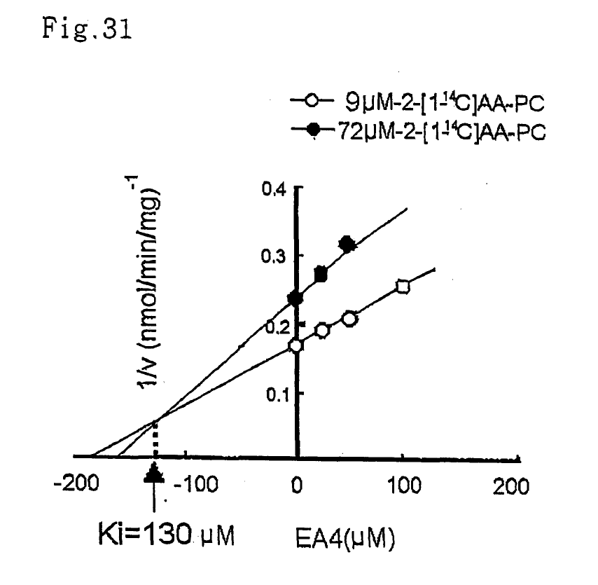

To verify the mechanism of inhibition, Dixon plot was constructed (FIG. 31). That show that the inhibition of rPLA₂ by EA4 is competitive, but not uncompetitive, with an inhibition constant of Ki=130 μM.

3) AA Production by rPLA₂ in use of rPLA₂ Inhibitor, EA4

To determine whether rPLA₂ was associated with production of AA in red blood cell, rPLA₂ inhibitor, EA4 was used.

According to Example 1-1), red blood cells from human and bovine, and L929 cells for control were prepared. After each type of cells were labeled with [³H]AA, those were washed 3 times with MEM medium to eliminate unincorporated [³H]AA. 50 μM of EA4 dissolved in DMSO was added and incubated for 20 min. and then 2 μM of A23187 compound also added at 37C.°. For control group, 2 μl of DMSO was added instead of inhibitor and A23187.

Cells were collected and centrifuged at the interval of 10 min. And the radioactivity was measured by scintillation counter.

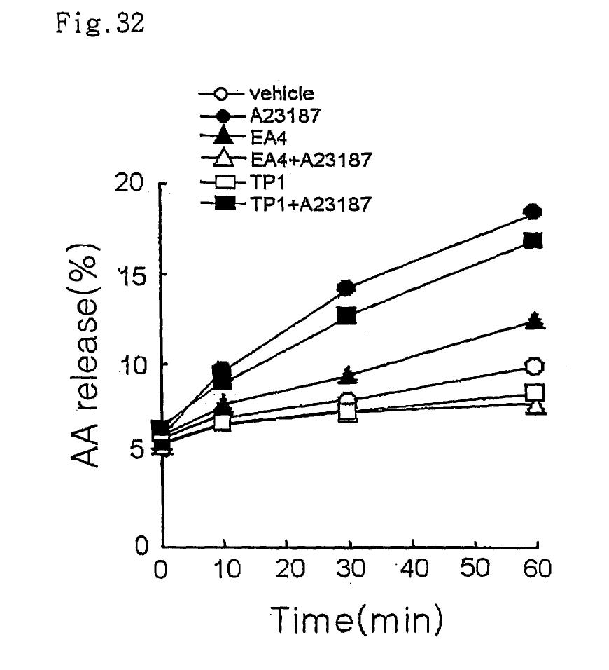

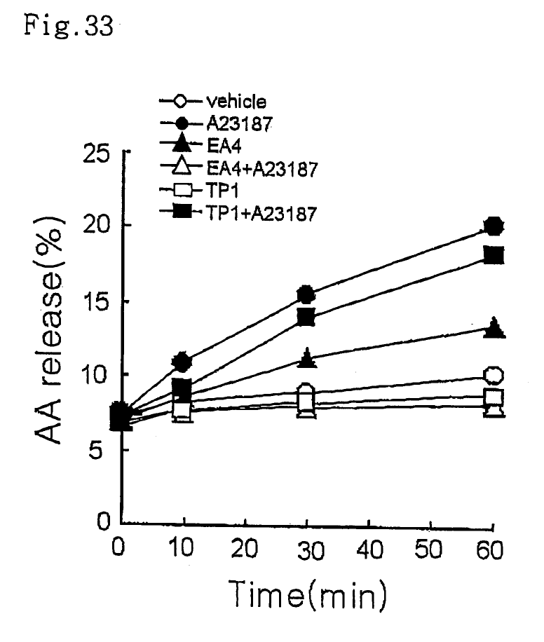

In human RBC (FIG. 32) and bovine RBC(FIG. 33), AA release by A23187 suggested in Example 1-(1) was significantly inhibited by EA4 and not inhibited by TP1.

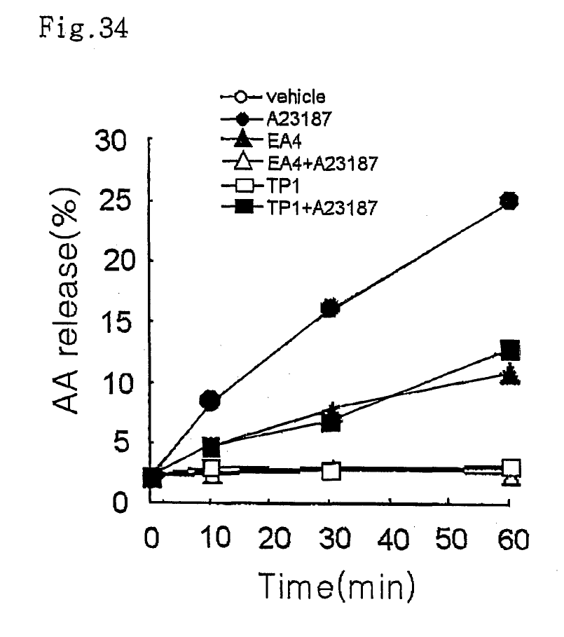

However, in L929 cells, AA release was inhibited by both EA4 and TP1 (FIG. 34). Since there exist cPLA₂ in L929 cells, whose activity is inhibited by both of EA4 and TP1.

Therefore, rPLA₂ is correlated to AA release induced by A23187 in RBC.

As mentioned above, the novel rPLA₂ enzyme of present invention showed the possibility of regulator in physiological mechanism associated with RBC, having Ca²⁺-dependent activity that alter phospholipids into arachidonic acids in bovine RBC.

The novel rPLA₂ antibody of the present invention, which binds 42 kDa protein rPLA₂ specifically and the effective rPLA₂ inhibitor EA4 of present invention can be used in pharmaceuticals for diagnosing, preventing and treating RBC-related disorders.

While the invention has been described with respect to the above specific embodiments, it should be recognized that various modification and changes may be made to the invention by those skilled in the art that also fall within the scope of the invention as described herein and in the claims below.

What is claimed is:

1. A cytosolic rPLA₂ enzyme derived from RBC cytosol characterized by the ability to produce arachidonic acid dependent on the presence of a calcium ion, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg.

2. The cytosolic rPLA₂ enzyme of claim 1, wherein said enzyme is not inhibited by DTT (dithiothreitol) or mepacrine (sPLA₂ inhibitor).

3. The cytosolic rPLA₂ enzyme of claim 1, wherein the enzyme is inhibited by AACOCF₃ (arachidonylfluoromethyl ketone).

4. The cytosolic rPLA₂ enzyme of claim 1, wherein the enzyme is activated by a divalent metal cation.

5. The cytosolic rPLA₂ enzyme of claim 4, wherein the divalent metal cation is selected from the group consisting of Zn²⁺, Fe²⁺, Cu²⁺, Sr²⁺, Ba²⁺, Mn²⁺, and Mg²⁺.

6. The cytosolic rPLA₂ enzyme of claim 1, wherein said enzyme is characterized in that it does not react with an anti-cPLA₂ antibody.

7. The cytosolic rPLA₂ enzyme of claim 1, wherein said enzyme is characterized in that it does not react with an anti-sPLA₂ antibody.

8. The cytosolic rPLA₂ enzyme of claim 1, wherein said enzyme is originated from human RBC.

9. The cytosolic rPLA₂ enzyme of claim 1, wherein said enzyme is originated from bovine RBC.

10. The cytosolic rPLA₂ enzyme of claim 1, wherein said enzyme has a molecular weight of 42 kDa determined by SDS-PAGE, an isoeletric point from 3.9 to 4.1, having maximum activity at a pH range from 9.5 to 10 and having a specific activity of 5.6 nM/min/mg.

11. A method for preparing a purified cytosolic rPLA₂ enzyme, comprising:
preparing a cytosolic fraction from red blood cells (RBCs); and
subjecting the cytosolic fraction to one or more column chromatography techniques selected from the group consisting of butyl-Toyopearl hydrophobic column chromatography, pheny-5PW hydrophobic HPLC column, DEAE-5PW HPLC column chromatography, Sephacryl S-300 gel filtration column chromatography, superose 12 gel filtration FPLC column chromatography, and Mono Q FPLC column chromatography, or a combination thereof, and
isolating the purified cytosolic rPLA₂ enzyme, wherein the purified rPLA₂ enzyme is characterized by the ability to produce arachidonic acid dependently to calcium ion, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg.

12. The method of claim 11, wherein the purified rPLA₂ enzyme is characterized by the ability to produce arachidonic acid dependently to calcium ion, having a molecular weight of 42 kDa determined by SDS-PAGE, an isoeletric point from 3.9 to 4.1, having maximum activity at a pH range from 9.5 to 10 and having a specific activity of 5.6 nM/min/mg

13. A method for producing an antibody to a rPLA₂ enzyme, comprising:
isolating the rPLA₂ enzyme, wherein the rPLA₂ is characterized by the ability to produce arachidonic acid dependent on the presence of calcium, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg;
providing an antigenic amount of the rPLA₂ enzyme to a host; and
isolating serum from the host, wherein the antibody to the rPLA₂ enzyme is present in the serum.

14. The method of claim 13, wherein the rPLA₂ is characterized by the ability to produce arachidonic acid dependent on the presence of calcium, having a molecular weight of 42 kDa determined by SDS-PAGE, an isoeletric point from 3.9 to 4.1, having maximum activity at a pH range from 9.5 to 10 and having a specific activity of 5.6 nM/min/mg

15. An anti-rPLA₂ enzyme specific antibody, wherein the antibody reacts with an rPLA₂ enzyme characterized by the ability to produce arachidonic acid dependent on the presence of calcium, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg.

16. The antibody of claim 15, wherein said antibody is characterized in that react with an rPLA₂ enzyme characterized by the ability to produce arachidonic acid dependent on the presence of calcium, having a molecular weight of 42 kDa determined by SDS-PAGE, an isoeletric point from 3.9 to 4.1, having maximum activity at a pH range from 9.5 to 10 and having a specific activity of 5.6 nM/min/mg.

17. The antibody of claim 15, wherein said antibody is characterized in that the antibody does not react with cPLA₂ or sPLA₂.

18. A pharmaceutical composition comprising:
a therapeutically effective amount of an antibody, wherein the antibody reacts with an rPLA₂ enzyme characterized by the ability to produce arachidonic acid in a calcium dependent manner, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg and a pharmaceutically acceptable carrier.

19. The pharmaceutical composition of claim 18, wherein the antibody reacts with an rPLA₂ enzyme characterized by the ability to produce arachidonic acid in a calcium dependent manner, having a molecular weight of 42 kDa determined by SDS-PAGE, an isoeletric point from 3.9 to 4.1, having maximum activity at a pH range from 9.5 to 10 and having a specific activity of 5.6 nM/min/mg.

20. A pharmaceutical composition comprising a therapeutically effective amount of EA4 (7-chloro-6-[4-(diethylamine)phenyl]-5,8-quinolinedione) compound and a pharmaceutically acceptable carrier.

21. A method of treating a disease caused or exacerbated by Ca²⁺ dependent release of arachidonic acid comprising:
administering to a subject an effective amount of an anti-rPLA₂ specific antibody, wherein the antibody reacts with an rPLA₂ enzyme characterized by the ability to produce arachidonic acid in a calcium dependent manner, having a molecular weight of about 42 kDa determined by SDS-PAGE, an isoeletric point from about 3.9 to about 4.1, having maximum activity at a pH range from about 9.5 to about 10 and having a specific activity of about 5.6 nM/min/mg and a pharmaceutically acceptable carrier, whereby a symptom of the disease is alleviated.

22. The method of claim 21, wherein the antibody reacts with an rPLA₂ enzyme characterized by the ability to produce arachidonic acid in a calcium dependent manner, having a molecular weight of 42 kDa determined by SDS-PAGE, an isoeletric point from 3.9 to 4.1, having maximum activity at a pH range from 9.5 to 10 and having a specific activity of 5.6 nM/min/mg.

23. A method of treating a disease caused or exacerbated by Ca²⁺ dependent release of arachidonic acid comprising:
administering to a subject in need thereof an effective amount of an rPLA₂ inhibitor and a pharmaceutically acceptable carrier, whereby a symptom of the disease is alleviated.

24. The method of claim 23, wherein the rPLA₂ inhibitor is EA4 (7-chloro-6-[4-(diethylamine)phenyl]-5,8-quinolinedione).
